(19) United States
(12) Patent Application Publication
(10) Pub. No.: US 2024/0197240 A1
(43) Pub. Date: Jun. 20, 2024

(54) ELECTRICAL IMPEDANCE TOMOGRAPHY BASED LIVER HEALTH ASSESSMENT

(71) Applicant: Gense Technologies Limited, Hong Kong (CN)

(72) Inventors: Russell Wade Chan, Kowloon (HK); Fedi Zouari, Tseung Kwan O (HK); Adrien Touboul, Wan Chai (HK); Luca Minciullo, Wan Chai (HK); Dipyaman Modak, Mong Kok (HK); Pak Heng Justin Chan, HK (CN)

(21) Appl. No.: 18/552,286

(22) PCT Filed: Mar. 23, 2022
(86) PCT No.: PCT/CN2022/082517

§ 371 Date: Sep. 25, 2023

(30) Foreign Application Priority Data
Mar. 23, 2021 (HK) 32021027899.2

(51) Int. Cl. A61B 5/00; A61B 5/0537

(52) U.S. Cl. CPC A61B 5/4244; A61B 5/0537; A61B 5/7264

(57) ABSTRACT

A computer-implemented method for liver health assessment, comprising: receiving EIT data associated with a liver of a subject; and processing the EIT data to determine a health condition of the liver of the subject.

### TECHNICAL FIELD

The invention relates to systems and methods for analyzing electrical impedance tomography (EIT) data for liver health assessment. The invention can be implemented as a diagnostic tool.

### BACKGROUND

Liver steatosis disease is a condition in which liver cells contain more than 5% fat. This condition is becoming increasingly common due to unhealthy food habits and sedentary lifestyle.

Furthermore, it is a silent disease with symptoms arising only at the later stages (e.g. fibrosis), many patients would be diagnosed at the advanced stages when fat accumulation, scarring and liver cell damage are irreversible. Existing diagnosing techniques are either expensive, harmful or both. Therefore, there is a need for relatively cost effective and non-invasive solutions for diagnosing Liver steatosis, or more generally, determining a health condition of a liver of the subject.

### SUMMARY OF THE INVENTION

In a first aspect, there is provided a method for prediction (optionally automated prediction) of liver biomarkers using a machine learning processing model trained on EIT derived measurements and anthropometrics. The method comprises of: a multi-frequency collection of voltage difference measurement by an EIT device; a 2D abdomen shape prior, an EIT solver to reconstruct frequency difference based images. A machine learning regression model to learn the functional relationship between EIT derived measurements and liver biomarkers. The method may be implemented by one or more computing devices.

In a second aspect, there is provided a (computer-implemented) method for determination or prediction (optionally automated determination or prediction) of liver biomarkers using a trained machine learning processing model (e.g., that has been trained on EIT derived measurements and anthropometrics), the method comprising one or more of: reconstructing frequency difference based images using a multi-frequency collection of voltage difference measurement by an EIT device and a (2D) abdomen shape prior; and training a machine learning processing model (e.g., regression model) based on determined relationship (e.g., functional relationship) between EIT derived measurements and liver biomarkers.

In a third aspect, there is provided a system for determination or prediction (optionally automated determination or prediction) of liver biomarkers using a trained machine learning processing model (e.g., that has been trained on EIT derived measurements and anthropometrics), the system comprising: an EIT device arranged to measure or a memory arranged to store a multi-frequency collection of voltage difference; a/the memory storing a 2D abdomen shape prior; one or more processors comprising an EIT solver to reconstruct frequency difference based images, and wherein the one or more processors operating a machine learning processing model (e.g., regression model) to learn the functional relationship between EIT derived measurements and liver biomarkers.

In a fourth aspect, there is provided a computer-implemented method for liver health assessment, comprising: receiving EIT data associated with a liver of a subject; and processing the EIT data to determine a health condition of the liver of the subject.

Optionally, the EIT data comprises multi-frequency EIT voltage data, which may be a pair of multi-frequency EIT voltage data.

Optionally, the processing comprises: processing the EIT data using a trained machine learning processing model to determine a property associated with a liver biomarker of the subject.

Optionally, the processing comprises: processing the EIT data using a trained machine learning processing model to determine a controlled attenuation parameter (CAP) value of the subject.

Optionally, the trained machine learning processing model comprises a regression model, which may be a linear regression model or a non-linear regression model.

Optionally, the regression model determines the controlled attenuation parameter (CAP) value of the subject based on a conductivity measure of the subject as determined from the EIT data and an anthropometric variable of the subject.

Optionally, the conductivity measure comprises a spatial average of the change in conductivity.

Optionally, the anthropometric variable comprises a waist circumference over height measure. Optionally, the anthropometric variable comprises age, height, weight, etc., of the subject.

Optionally, the processing further comprises: performing an image reconstruction operation prior to processing the EIT data using the trained machine learning processing model.

Optionally, the image reconstruction operation comprises: determining change in conductivity images based on processing the EIT data with reference to abdomen shape prior or reference data. The abdomen shape prior or reference data may be a CT image data of a reference abdomen. The use of the abdomen shape prior or reference data improves accuracy in the processing of the EIT data for determining conductivity or conductivity change.

Optionally, the processing further comprises: performing a post-processing operation after the image reconstruction operation and prior to processing the EIT data using the trained machine learning processing model.

Optionally, the post-processing operation comprises: segmenting liver regions from the change in conductivity images; and determining a spatial average of the change in conductivity.

Optionally, segmenting the liver regions comprises: segmenting the liver regions from the change in conductivity images with reference to a liver shape prior or reference data. The liver shape prior or reference data may be a CT image data of a reference liver. The use of the liver shape prior or reference data improves accuracy in the processing of the EIT data for determining conductivity or conductivity change.

In a fifth aspect, there is provided a system for liver health assessment, comprising one or more processors arranged to: receive multi-frequency EIT voltage data associated with a liver of a subject; and process the multi-frequency EIT voltage data using a trained machine learning processing model to determine a property associated with a liver biomarker of the subject, so as to determine a health condition of the liver of the subject. The one or more processors may be arranged to perform the method of the fourth aspect. Optionally the system further comprises a display for displaying the processing results.

In a sixth aspect, there is provided a non-transitory computer-readable medium comprising instructions which, when executed by one or more processors, causes the one or more processors to perform the computer-implemented method for liver health assessment of the fourth aspect.

Some embodiments of the invention provide a reliable pipeline/system/method for the study/diagnosis/characterizing/screening of liver steatosis. The invention may be extended to the diagnosis of other liver illness or conditions.

Other features and aspects of the invention will become apparent by consideration of the detailed description and accompanying drawings. Any feature(s) described herein in relation to one aspect or embodiment may be combined with any other feature(s) described herein in relation to any other aspect or embodiment as appropriate and applicable.

### BRIEF DESCRIPTION OF THE DRAWINGS

Embodiments of the invention will now be described, by way of example, with reference to the accompanying drawings in which:

FIG. 1 is a flow chart showing a method for analyzing Electrical Impedance Tomography (EIT) based liver data (e.g., multi-frequency) in one embodiment of the invention.

FIG. 2 is a schematic diagram illustrating a method for determining CAP value based on frequency-difference EIT in one embodiment of the invention.

FIG. 3 is a diagram that illustrates decomposition of the total variance of the estimator into the variance due to repetition and the variance due to measurement noise with an area of reference for N_{R}=7.

FIG. 4 is a graph showing the performance of the fdEIT based method embodiments (fdEIT, fdEIT+spectral unmixing) in determining CAP value.

FIG. 5 contains graphs showing average CAP values across healthy population (H) and non-healthy (NH) as classified by Fibroscan CAP, wherein (A) represents the Fibroscan values, (B) represents fdEIT embodiment of the invention and (C) represents fdEIT+spectral unmixing embodiment of the invention.

### DETAILED DESCRIPTION

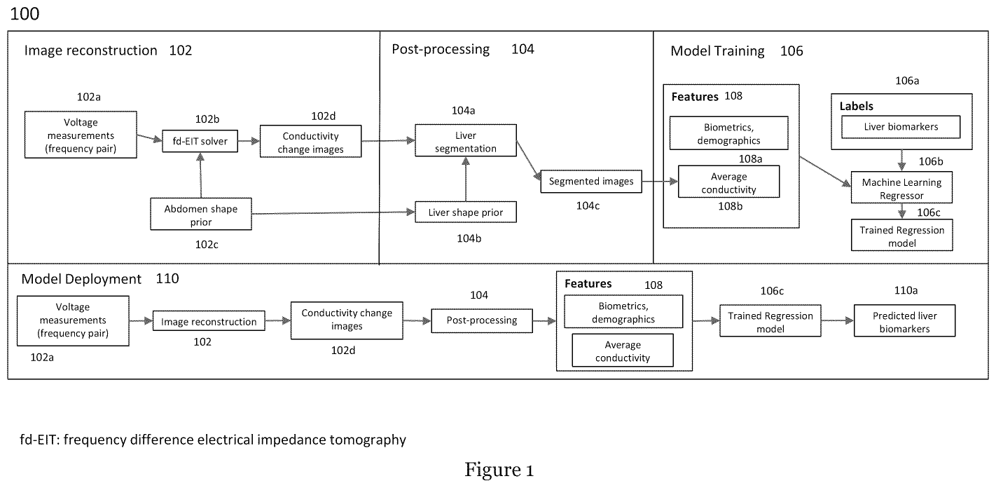

One embodiment of the invention is described in FIG. 1. First, an abdomen shape prior should be prepared (102c). This consists of a 2D abdomen mesh, where each element contains the representative conductivity value for the corresponding tissue type. As a result, this step outlines the organ shapes within the abdomen. Multi-frequency EIT voltage data for the abdomen should be gathered (102a). Then, a relative imaging EIT solver (102b) is used, in this embodiment frequency pairs are used for relative imaging. The previously defined abdomen shape mesh is integrated as a penalization term during image reconstruction to encourage the reconstruction of images close to the desired internal organ outlines. The EIT reconstruction cost function can then be written as follows:

∥vₘ−v_{c}(σ₀)∥+λ(α∥L^{T}L(σ−σ₀)∥+(1−α)∥(J^{T}J)^{β}_{diag}σ∥,

where vₘ is the measured voltage, σ₀ is the initial conductivity, v_{c} is the voltage computed based on the initial conductivity, λ is a regularization parameter, α is the penalization weight. L is a matrix such that the prior vectors are in its null-space.

The reconstructed image (102d) is passed through a post-processing stage (104), where a rough liver outline is segmented (104a) using the binary mask defined by the liver region in the abdomen shape prior (104b), resulting in the segmented image (104c). Next, a collection of such segmented images should be obtained together with anthropometric data, such as weight, height, waist circumference, etc. (108a). Multiple order statistics are extracted from the segmented images (108b) and concatenated with anthropometric data, leading to 1D feature vectors (108). Paired labels (such as CAP scores for fatty liver, steatosis grades or fibrosis indicators) should be then acquired (106a). The paired data can then be fed into a machine learning regression model (106b). The model will be trained to predict the liver biomarkers given a segmented EIT image and anthropometrics.

When deploying the model (110) a new pair of multi-frequency EIT voltage data should be collected. Next, image reconstruction (102), post-processing (104) and feature extraction/concatenation (108) should be performed, obtaining a new 1D feature vector. The trained model (106c) should then be applied to the latter to yield a predicted liver biomarker (110a).

The above method embodiment can be implemented using the system disclosed in U.S. Non-Provisional patent application Ser. No. 16/976,542, the entire contents of which is incorporated herein by reference (by choosing an appropriate form factor, the wrist band disclosed in U.S. Ser. No. 16/976,542 can be implemented as a wearable EIT band/belt/harness for liver EIT data collection).

In the following specific embodiments of the invention are provided.

The inventors of the present invention have devised, through research, experiments, and trials, that invasive liver biopsy is the medical standard to diagnose nonalcoholic fatty liver disease (NAFLD). The inventors of the present invention have devised, through research, experiments, and trials, that non-invasive procedures based on ultrasound-based devices and vibration-controlled transient elastography (VCTE) could also be used. These ultrasound-based devices measure liver elasticity to infer hepatic steatosis in NAFLD, quantified using the CAP (controlled attenuation parameter).

Some embodiments of the invention apply frequency sweeping to predict CAP from cross-sectional EIT measurement across the liver, with both a frequency difference and a spectral unmixing model.

Electrical impedance tomography (EIT) is a noninvasive imaging method based on measuring electrical impedance of living tissue (bio-impedance). A small electrical current (usually approx. 1 mA, which does not affect normal physiology) is applied into the body through a belt of electrodes at frequencies ranging typically from 1 kHz to 1 MHz. This electrical current induces an electrical potential, that is measured at each electrode. Using this input, a map of the conductivity inside the body is reconstructed. In some embodiments of the invention these changes in conductivity are used to predict CAP values.

The EIT reconstruction problem is now presented.

### Forward Problem

Assume that a current is injected in the body through a source electrode and a sink electrode. If the conductivity inside the body is known, it would be possible to compute the electrical potential inside the body using Maxwell's equations

$$\{ \begin{matrix}∇ \cdot ( σ ∇ V ) = 0 inside the body \\ \int σ \frac{\partial V}{\partial n} = \pm I at the \frac{source}{sink} electrode \\ \frac{\partial V}{\partial n} = 0 at the other boundaries\end{matrix} \qquad ( 1 )$$

where σ denotes the conductivity inside the body and V the electrical potential. This equation can be simulated with good precision.

### Inverse Problem

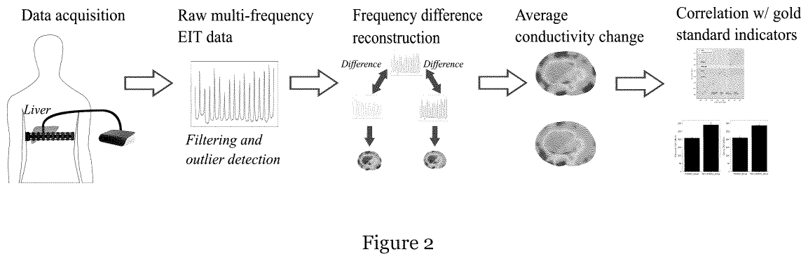

The main theoretical challenge of EIT is that neither the conductivity a nor the potential V inside the body is known. Instead, they must be recovered from boundary measurements Vₘₑₛ, leading to the so-called inverse problem. Some embodiments of the invention try to recover only the change in conductivity between two frequencies (see FIG. 2). Using linear approximation, this type of reconstruction is easier than absolute imaging. The algorithm used in some embodiments of the invention is the linearized least-square algorithm with Kotre regularization and prior information extracted from CT scans. By denoting the forward operator that maps the conductivity to the measured potential using Vₘₑₛ=F(σ), the change in potential can be expressed as.

Vₘₑₛ−V_{mes,0}=F(σ)−F(σ₀)≃∇F(σ₀)·(σ−σ₀)

This relationship is not enough to recover σ from boundary measurement, because of the ill-posedness of the problem. A regularization term and a prior information are incorporated in some embodiments, so that the discretized final expression of the conductivity is

(σ−σ₀)=(∇F^{T}∇F+λ((1−α)R^{p}+αWₓ))⁻¹·∇F^{T}·(Vₘₑₛ−V_{mes,0})

where R is the Kotre diagonal sensitivity matrix, α and p two parameters and Wₓ a matrix incorporating prior information.

Tests has been performed using the method embodiments of the invention.

A total of 11 human subjects including, 3 females and 8 males, from 20 to 65 years old with a waist circumference from 71 cm to 110 cm are subjected to the tests. Clinical demographics and physical characteristics of these individuals are collected, namely gender, BMI, age, waist circumference, height, weight, and liver disease history (if any). The human subjects have no co-existing liver diseases.

The subjects first undergo 5-10 minutes liver FibroScan session (Echosens, France) to obtain the CAP value. EIT examination is then performed with a portable system (Gense Technologies Limited) composed of a 15.2×11.0×4.4 cm³ acquisition console and a 16-electrode belt. The console consists of a power management module, with a current generator providing alternating current at frequencies ranging from 10 KHz to 1 MHz, a data acquisition module for potential difference measurement, and a control and output module for module coordination, data processing and cloud-server communication. The position of the electrode belt targeted the upper abdominal region, as indicating by the bottom boundary of the ribcage.

The conductivity is reconstructed using a custom version of the pyEIT python library. The changes in conductivity between two pairs of frequency (28.75 kHz-20.00 kHz) and (28.75 kHz-25.00 kHz) are computed by averaging the conductivity map of the difference over the region of interest (ROI) covering the whole liver. Both the prior and region of interest are extracted from a CT scan image.

To extract more information from the frequency-difference curve, some embodiments of the invention additionally apply the spectral unmixing method for conductivity. The idea relies on the fact that if the change in conductivity with respect to (w.r.t) frequency is small, then the shape of the measured voltage over frequency is given by a linear combination of the shape of the conductivity changes over frequency. For example, the conductivity change over frequency can be approximated by its Taylor expansion:

$$σ ( x , ω ) - σ ( x , {ω}_{0} ) = \frac{\partial σ ( x , {ω}_{0} )}{\partial ω} ( ω - {ω}_{0} ) + \frac{{\partial }^{2} σ ( x , {ω}_{0} )}{\partial {ω}^{2}} \frac{{( ω - {ω}_{0} )}^{2}}{2} + O ( {( ω - {ω}_{0} )}^{2} ) .$$

The measured potential is equal to Vₘₑₛ(ω)=F(σ(ω)), which we approximate by the discrete operator Vₘₑₛ(ω)≃\tilde{F}(σ(x_{N}, ω)), where x_{N} is vector of points that discretizes the domain. The difference of potential can be approximated as follows:

$${V}_{mes} ( ω ) - {V}_{mes} ( {ω}_{0} ) = ( ∇ \tilde{F} ( ( σ ( {x}_{N} , {ω}_{0} ) ) ) \cdot \frac{\partial σ ( {x}_{N} , {ω}_{0} )}{\partial ω} ) ( ω - {ω}_{0} ) + ( ∇ \tilde{F} ( ( σ ( {x}_{N} , {ω}_{0} ) ) ) \cdot \frac{{\partial }^{2} σ ( {x}_{N} , {ω}_{0} )}{\partial {ω}^{2}} ) \frac{{( ω - {ω}_{0} )}^{2}}{2} + o ( {( ω - {ω}_{0} )}^{2} ) + o ( ‖ σ ( {x}_{N} , ω ) - σ ( {x}_{N} , {ω}_{0} ) ‖ ) \qquad ( 2 )$$

and, by neglecting the last two terms (in particular, the Hessian of F must be small), the following can be obtained

$$\frac{\partial V}{\partial ω} ( {ω}_{0} ) ≃ ∇ \tilde{F} ( {( σ ( {x}_{k} , {ω}_{0} ) )}_{k ∈ \{ 1 , … , N \}} ) \cdot {( \frac{\partial σ ( {x}_{k} , {ω}_{0} )}{\partial ω} )}_{k ∈ \{ 1 , … N \}} \\ \frac{{\partial }^{2} V}{\partial {ω}^{2}} ( {ω}_{0} ) ≃ ∇ \tilde{F} ( {( σ ( {x}_{k} , {ω}_{0} ) )}_{k ∈ \{ 1 , … , N \}} ) \cdot {( \frac{{\partial }^{2} σ ( {x}_{k} , {ω}_{0} )}{\partial {ω}^{2}} )}_{k ∈ \{ 1 , … N \}}$$

The relationship between

$$\frac{\partial V}{\partial ω} ( {ω}_{0} ) ( resp . \frac{{\partial }^{2} V}{\partial {ω}^{2}} ( {ω}_{0} ) ) and \frac{\partial σ ( {x}_{k} , {ω}_{0} )}{\partial ω} ( resp . \frac{{\partial }^{2} σ ( {x}_{k} , {ω}_{0} )}{\partial {ω}^{2}} )$$

is the same as the relationship between ΔV and Δσ. Thus, the same method can be used to reconstruct Δσ and reconstruct

$$\frac{\partial σ ( {x}_{k} , {ω}_{0} )}{\partial ω} ( resp . \frac{{\partial }^{2} σ ( {x}_{k} , {ω}_{0} )}{\partial {ω}^{2}} ) .$$

Assume that the parts of the body that cause first order and second order change are different, and thus isolating them would provide more specific information. Note that in this embodiment the method is presented with an expansion up to two orders. However, the method can be extended to higher orders, provided the conductivity changes have enough derivatives. It can also be extended to any functional basis/free family decomposition. Assuming that M tissue types are contributing to the conductivity change and they each have a conductivity variation of Δσᵢ(ω), then, under the linearity assumption (2):

ΔV(ω)=Σᵢ₌₁^{M}aᵢΔσᵢ(ω)+ϵ,

where ϵ is an error term and aᵢ the impact of the change due to organ i. The aᵢ would reconstruct the image at the specific position of the organ i, in a similar fashion as linear spectral unmixing.

The Δσᵢ(ω) can be obtained by tabulated values and the coefficient can be estimated by classic linear mixed model methods (Error! Reference source not found.).

TABLE I
Summary of the recovered map w.r.t the given inputs

| | | | | |
|---|---|---|---|---|
| Input | ΔV | $\frac{\partial V}{\partial ω}$ | $\frac{{\partial }^{2} V}{\partial {ω}^{2}}$ | aᵢ (coefficient due to organ i) |
| Recovered map | Δσ | $\frac{\partial σ}{\partial ω}$ | $\frac{{\partial }^{2} σ}{\partial {ω}^{2}}$ | Δσᵢ (change at organ i) |

After the model for the CAP is constructed, more EIT data (without measuring the VCTE CAP) are acquired to minimize the variance of the prediction or determination. For each frequency acquisition, the duration of the acquisition and the number of repetitions can be freely chosen in different embodiments. Assume that the data acquired at the frame i for the repetition j provides an observation of the CAP of

[symbol]

_{i,j}=CAP+ϵ_{F,i,j}+ϵ_{R,j}

where CAP is the true value of the CAP, ϵ_{F,i,j} is the measurement error (i.e., due to sensor noise) and ϵ_{R,j} is the repetition error (the subject does not repeat exactly the same pattern). Deonte by N_{F} the number of frames acquired per repetition and by N_{R} the number of repetitions; in order to estimate the CAP, we use the Monte-Carlo average over all the frames

$$\overline{CAP} = \frac{1}{{N}_{R}} {\sum }_{j = 1}^{{N}_{R}} \frac{1}{{N}_{F}} {\sum }_{i = 1}^{{N}_{F}} {i , j}_{} .$$

Assuming that the ϵ_{F,i,j}, ϵ_{R,j} are independent random variables and the errors ϵ_{F,i,j} (resp. ϵ_{R,j}) share the same finite variance σ_{F}² (resp. σ_{R}²), we get, by the central limit theorem,

$$Var ( \overline{CAP} ) ≃ \frac{{σ}_{F}^{2}}{{N}_{F} {N}_{R}} + \frac{{σ}_{R}^{2}}{{N}_{R}} ,$$

when N_{R} and N_{F} are large enough. Some embodiments of the invention model duration of one acquisition by the product of the number of frames N_{F} by the duration of the acquisition of one frame t_{F} plus a setup time t_{S}. The total duration of all the acquisitions is then T=N_{R}(N_{F}t_{F}+t_{S}).

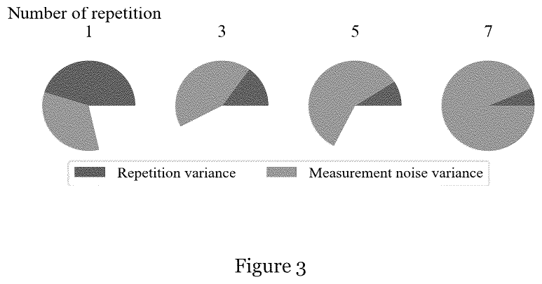

By fixing the total time T=T₀ and the previous equations, some embodiments of the invention decompose the total variance into the part due to measurement noise and the part due to repetition (FIG. 3). The best theoretical value can be computed, and are

$${N}_{F} = \sqrt{\frac{{t}_{S}}{{t}_{F}}} \frac{{σ}_{F}}{{σ}_{R}} and {N}_{R} = \frac{{T}_{0}}{\frac{\sqrt{{t}_{S} {t}_{F}} {σ}_{F}}{{σ}_{R}} + {t}_{S}} .$$

Finally, the values are chosen by rounding off the obtained real values. For a setup time of t_{S}=5 s and a total acquisition time of T₀=30 s per frequency, the optimal number of repetitions is N_{R}=3 and the acquisition time is N_{F}t_{F}=5 second.

The test results are as follows.

### Linear Regression with Simple Difference

TABLE II
Results of the linear regression of the
CAP vs change in conductivities and WoH

| | | | |
|---|---|---|---|
| Dep. Variable: | CAP | R² | 0.935 |
| F-statistic: | 57.42 | Adjusted-R² | 0.919 |
| Features (normalized by std) | Coeff | Standard error | P > \|t\| |
| dC_{28-22 kHz} | −18.4983 | 6.119 | 0.016 |
| dC_{28-25 kHz} | −19.6607 | 6.248 | 0.014 |
| WoH | 33.4490 | 1.393 | 0.000 |
| Mean Average Percentage Error | 6% | No. of | 11 |
| (Leave One Out) | | Observations | |

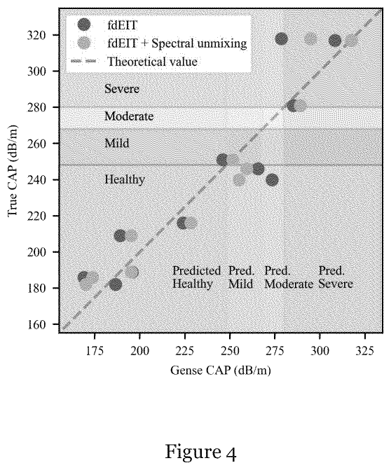

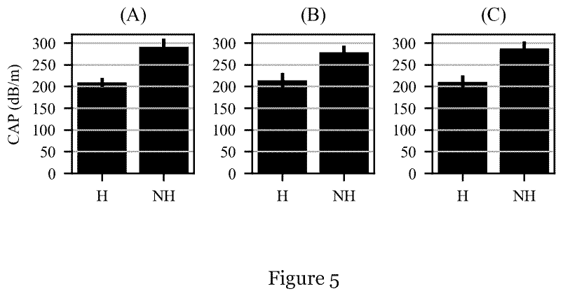

In some embodiments the CAP is predicted by a linear regression using as features the spatial average of the change in conductivity dC_{28.75 kHz-22 kHz}, dC_{28.75 kHz-25 kHz} and the anthropometric variable WoH (waist circumference over height). All the variables are significant under a student t-test at level 0.05%. The coefficient of determination is 0.932 and the adjusted one is 0.919 (Error! Reference source not found.). The predicted CAP seems to be able to classify the liver state based on the VCTE CAP, (sensitivity=0.75 and a specificity=0.71, the subject being incorrectly classified being mostly close to the threshold, FIGS. 4 and 5). The regression keeps a good homoscedasticity, even for high value of the CAP, suggesting that the model embodiments can generalize well across a wide range of values.

As shown in Table III, including both conductivity and WoH improves the quality of the regression, compared to using solely one of the two. The coefficients of the linear regression using normalized data being of the same order of magnitude, thus it can be considered that they have the same importance in the prediction.

TABLE III
Coefficient of determination for different models.

| Features used | | dC_{28-22 kHz}& | dC_{28-22 kHz}& |
|---|---|---|---|
| in the linear | | dC_{28-25 kHz}& | dC_{28-25 kHz} & |
| regression | WoH | intercept. | WoH |
| R² | 0.425 | 0.444 | 0.935 |
| R²-adjusted | 0.425 | 0.305 | 0.919 |

Linear Regression with Unmixed Polynomial Coefficients

As explained with reference to classic frequency-difference mentioned above, the first order and second order variation of V w.r.t ω are used to reconstruct the conductivity. For each subject the α and β are estimated using least-squares verifying Vₘₑₛ(ω)−Vₘₑₛ(ω₀)≃α(ω−ω₀)+β(ω−ω₀)² and are used to reconstruct an image. The average conductivity in the liver area dC_{α} and dC_{β} is computed, and these values are used as regressors. The polynomial model provides a good approximation of the change in potential between 10 kHz and 35 kHz (MAPE of 4%). Their performances in the predicted cap are similar to the simple difference, with an adjusted R-squared of 0.914 with WoH (FIGS. 4 and 5).

The above results suggest a strong correlation between the difference of conductivity across two frequency pairs and the CAP measured by VCTE, in addition to the already observed correlation with the waist circumference over height (WoH) This correlation can be explained by the conductivity change w.r.t. the fat content in liver tissue and is captured by EIT. A shape prior (reference data) is used to focus on the liver region.

The use of a self-administrable EIT device for obtaining the results (instead of a VCTE) enables a more affordable measure with a real-time result, without needing the help of a trained professional for acquisition.

The spectral unmixing method embodiments provide results similar to classic fdEIT (adj. R-squared of 0.914), confirming the validity of the approach. Without wishing to be bound by theory, the methods of the present invention may be applied to other organs (such as kidney). A promising direction is the use tabulated/measured frequency changes instead of a polynomial basis, that could allow to more precisely target specific organs.

The above embodiments of the invention demonstrate that multi-spectral electrical impedance tomography (EIT) can predict clinical-standard controlled attenuation parameter (CAP) in patients with or without nonalcoholic fatty liver disease (NAFLD) using waist over height (WoH) anthropometric as complementary information. Some of the above embodiments of the invention also incorporates a spectral unmixing method to estimate CAP from multi-spectral EIT by matching the coefficient of a functional decomposition. Some of the above embodiments of the invention provides an algorithm for determining the optimal number of repetitions (N_{R}=3) and acquisition time (N_{F}tₛ=5 s) that minimizes the error and maximizes the accuracy under a constraint of time.

It will be appreciated that where the methods and systems of the invention are either wholly implemented by computing system or partly implemented by computing systems then any appropriate computing system architecture may be utilized. This will include stand-alone computers, network computers, dedicated or non-dedicated hardware devices. Where the terms “computing system” and “computing device” are used, these terms are intended to include (but not limited to) any appropriate arrangement of computer or information processing hardware capable of implementing the function described.

It will be appreciated that numerous variations and/or modifications may be made to the invention as shown in the specific embodiments to provide other embodiments of the invention. The described embodiments of the invention should therefore be considered in all respects as illustrative, not restrictive.

While some of the systems and methods embodiments are described with reference to liver, it is envisaged that the systems and methods embodiments can be used to process data of other organs. This may involve changing the prior 102c, 104b, and the model 106 to suit the organ of interest.

The invention has provided a computer-implemented method for liver health assessment, comprising: receiving EIT data associated with a liver of a subject; and processing the EIT data to determine a health condition of the liver of the subject. Optionally, the EIT data comprises multi-frequency EIT voltage data, which may be a pair of multi-frequency EIT voltage data. Optionally, the processing comprises: processing the EIT data using a trained machine learning processing model to determine a property associated with a liver biomarker of the subject. Optionally, the processing comprises: processing the EIT data using a trained machine learning processing model to determine a controlled attenuation parameter (CAP) value of the subject. Optionally, the trained machine learning processing model comprises a regression model, which may be a linear regression model or a non-linear regression model. Optionally, the regression model determines the controlled attenuation parameter (CAP) value of the subject based on a conductivity measure of the subject as determined from the EIT data and an anthropometric variable of the subject. Optionally, the conductivity measure comprises a spatial average of the change in conductivity. Optionally, the anthropometric variable comprises a waist circumference over height measure. Optionally, the anthropometric variable comprises age, height, weight, etc., of the subject. Optionally, the processing further comprises: performing an image reconstruction operation prior to processing the EIT data using the trained machine learning processing model. Optionally, the image reconstruction operation comprises: determining change in conductivity images based on processing the EIT data with reference to abdomen shape prior or reference data. The abdomen shape prior or reference data may be a CT image data of a reference abdomen. The use of the abdomen shape prior or reference data improves accuracy in the processing of the EIT data for determining conductivity or conductivity change. Optionally, the processing further comprises: performing a post-processing operation after the image reconstruction operation and prior to processing the EIT data using the trained machine learning processing model. Optionally, the post-processing operation comprises: segmenting liver regions from the change in conductivity images; and determining a spatial average of the change in conductivity. Optionally, segmenting the liver regions comprises: segmenting the liver regions from the change in conductivity images with reference to a liver shape prior or reference data. The liver shape prior or reference data may be a CT image data of a reference liver. The use of the liver shape prior or reference data improves accuracy in the processing of the EIT data for determining conductivity or conductivity change. The invention has also provided a system and a non-transitory computer-readable medium for implementing the above computer-implemented method.

1. A computer-implemented method for liver health assessment, comprising:
receiving EIT data associated with a liver of a subject; and
processing the EIT data to determine a health condition of the liver of the subject.

2. The computer-implemented method of claim 1, wherein the EIT data comprises multi-frequency EIT voltage data, which may be a pair of multi-frequency EIT voltage data.

3. The computer-implemented method of claim 1 or 2, wherein the processing comprises:
processing the EIT data using a trained machine learning processing model to determine a property associated with a liver biomarker of the subject.

4. The computer-implemented method of claim 1 or 2, wherein the processing comprises:
processing the EIT data using a trained machine learning processing model to determine a controlled attenuation parameter (CAP) value of the subject.

5. The computer-implemented method of claim 4, wherein the trained machine learning processing model comprises a regression model, which may be a linear regression model or a non-linear regression model.

6. The computer-implemented method of claim 5, wherein the regression model determines the controlled attenuation parameter (CAP) value of the subject based on a conductivity measure of the subject as determined from the EIT data and an anthropometric variable of the subject.

7. The computer-implemented method of claim 6, wherein the conductivity measure comprises a spatial average of the change in conductivity.

8. The computer-implemented method of claim 7, wherein the anthropometric variable comprises a waist circumference over height measure.

9. The computer-implemented method of any one of 4 to 8, wherein the processing further comprises:
performing an image reconstruction operation prior to processing the EIT data using the trained machine learning processing model.

10. The computer-implemented method of claim 9, wherein the image reconstruction operation comprises:
determining change in conductivity images based on processing the EIT data with reference to abdomen shape prior or reference data.

11. The computer-implemented method of claim 10, wherein the processing further comprises:
performing a post-processing operation after the image reconstruction operation and prior to processing the EIT data using the trained machine learning processing model.

12. The computer-implemented method of claim 11, wherein the post-processing operation comprises:
segmenting liver regions from the change in conductivity images; and
determining a spatial average of the change in conductivity.

13. The computer-implemented method of claim 12, wherein segmenting the liver regions comprises:
segmenting the liver regions from the change in conductivity images with reference to a liver shape prior or reference data.

14. A system for liver health assessment, comprising one or more processors arranged to:
receive multi-frequency EIT voltage data associated with a liver of a subject; and
process the multi-frequency EIT voltage data using a trained machine learning processing model to determine a property associated with a liver biomarker of the subject, so as to determine a health condition of the liver of the subject.

15. A non-transitory computer-readable medium comprising instructions which, when executed by one or more processors, causes the one or more processors to perform the computer-implemented method for liver health assessment of any of claims 1 to 13.
